(19) United States
(12) Patent Application Publication
(10) Pub. No.: US 2004/0034107 A1
(43) Pub. Date: Feb. 19, 2004

(54) Ubiquinone Qn for the treatment of pain

(75) Inventor: Franz Enzmann, Bad Homburg (DE)

Correspondence Address: JACOBSON HOLMAN PLLC, 400 SEVENTH STREET N.W., SUITE 600, WASHINGTON, DC, 20004, US

(73) Assignee: MSE Pharmazeutika GmbH, Bad Homburg (DE)

(21) Appl. No.: 10/424,987

(22) Filed: Apr. 29, 2003

Related U.S. Application Data
(63) Continuation of application No. 09/890,276, filed on Aug. 10, 2001, now abandoned.
Application No. 09/890,276 is a 371 of international application No. PCT/EP00/01011, filed on Feb. 9, 2000.

(30) Foreign Application Priority Data
Feb. 11, 1999 (DE) 199 05 879.2

(51) Int. Cl. A61K 31/12

(52) U.S. Cl. 514/690

(57) ABSTRACT

Ubiquinone Qₙ or ubiquinone Qₙ precursors can be used for the treatment of pain.

Ubiquinones are prenylated quinones which are wide-spread in the animal and vegetable kingdoms. They are derivatives of 2,3-dimethoxy-5-methyl-1,4-benzoquinone having linearly linked isoprene units in the 6-position. Depending on the number of isoprene units, the ubiquinones are designated as Q-1, Q-2, Q-3 etc. In most mammals including humans, Q-10 (2,3-dimethoxy-5-methyl-6-decaprenyl-1,4-benzoquinone) is prevailing. Ubiquinones serve as electron carriers in the respiratory chain, and they participate in the cyclic oxidation and reduction of substrates in the citric acid cycle. Ubiquinones Qₙ represent a precondition of the energy supply to all cells. The oxidative stress which arises, inter alia, from a high oxygen consumption causes damage to the membranes of mitochondria and cells which result in acute or degenerative disorders of the nervous system. The nervous system has a very high energy demand for the signal transduction by membrane potential build-up, ion-channel control, as well as by neuropeptide and neurotransmitter vesicle formation.

Ubiquinone Q-10 (also referred to as coenzyme Q-10) has previously been used in the therapy of heart diseases.

Surprisingly, it has now been found that ubiquinone Qₙ and ubiquinone Qₙ precursors can be used for the treatment of pain. Thus, they can also be used in methods for the preparation of agents for the treatment of pain.

The term “ubiquinone Qₙ precursors” refers to compounds which are converted to ubiquinone Qₙ in the body. These include, on the one hand, the ubihydroquinones, which are in an equilibrium with the ubiquinones, as well as simple esters of the ubihydroquinones with short-chained carboxylic acids having from 1 to 10 carbon atoms, for example, acetate, propionate or butyrate esters. These precursors are converted to the corresponding ubiquinones after the application thereof.

Ubiquinone Q-10 is preferably used because this is the main ubiquinone in humans.

According to the invention, there can be treated, in particular, pain caused by a disturbance in the stimulus conduction in the nerves, and/or are out of proportion with the external cause. This is pain for which either there is no external cause, or an excessive signal is produced upon a minor cause and under oxidative stress conditions of the nerves.

The substances to be used according to the invention can be preferably employed for the treatment of pain which is caused by migraine, dialysis, herpes zoster, cancer, diabetic neuropathy, or generalized pain conditions. The treatment can be done by administration in oral, parenteral, local, inhalative or intranasal form. The kind of administration must be adapted to the pain condition to be treated. Thus, for example, it has been found that migraine can be treated even with high doses of Q-10 only with very limited success when oral administration is used. However, when used in the form of oral and nasal sprays, small amounts are sufficient for a fast and effective elimination of migraine pain. Good results were achieved already with doses of about 20 mg.

In the case of herpes zoster, even the local application of Q-10 in the form of creams or gels has even proven useful. It is critical that sufficient amounts of the ubiquinones or ubiquinone precursors arrive at the place in which the pain caused by disturbances of the signal transmission of the nerve system has its origin.

By combination with lung surfactant factor (pulmonary surfactant factor) as described in WO 08/35660, this effect can even be enhanced.

In kidney dialysis patients, the administration of ubiquinone or its precursors before the dialysis causes the dialytic procedure to proceed in a tolerable way.

Due to the low dose which is already effective for treating migraine pain, the ubiquinone Qₙ or its precursors are preferably used in the form of a spray, preferably a nasal spray, according to the invention.

In principle, the single dose may be as high as 1,000 mg.

Ubiquinones are lipophilic substances which are virtually insoluble in water. However, a particularly high effectiveness was found when the ubiquinone Qₙ or its precursors are in aqueous dispersion. Aqueous colloidal dispersions are particularly preferred. The preparation of the corresponding dispersions is described in WO 95/05164 and in the related DE-A-43 27 063.

1. Use of ubiquinone Qₙ or ubiquinone Qₙ precursors for the treatment of pain.

2. The use according to claim 1, characterized in that said ubiquinone Qₙ is ubiquinone Q-10.

3. The use according to any of claims 1 or 2, characterized in that said pain is caused by migraine, dialysis, herpes zoster, cancer, diabetic neuropathy, or generalized pain conditions.

4. The use according to any of claims 1 to 3, characterized in that the treatment is done by administration in oral, parenteral, local, inhalative or intranasal form.

5. The use according to claim 4, characterized in that the administration is in the form of a spray.

6. The use according to claim 5, characterized in that the administration is in the form of a nasal spray.

7. The use according to any of claims 1 to 6, characterized in that said ubiquinone Qₙ or ubiquinone Qₙ precursor is in an aqueous dispersion.

8. The use according to claim 7, characterized in that said ubiquinone Qₙ or ubiquinone Qₙ precursor is in an aqueous colloidal dispersion.
